(19) United States
(12) Patent Application Publication
(10) Pub. No.: US 2008/0113868 A1
(43) Pub. Date: May 15, 2008

(54) Colorant compositions and their use as pH sensitive color indicators

(76) Inventors: Patrick D. Moore, Pacolet, SC (US); Lacey D. Hilliard, Spartanburg, SC (US); Michael A. Valenti, Greenville, SC (US)

Correspondence Address: Legal Department (M-495), P.O. Box 1926, Spartanburg, SC, 29304, US

(21) Appl. No.: 11/595,450

(22) Filed: Nov. 9, 2006

(51) Int. Cl. D06P 5/13; A01N 25/00; C07C 211/44; C07C 63/331; A61K 47/16

(52) U.S. Cl. 504/358; 8/403; 564/315; 562/480; 514/788

(57) ABSTRACT

This invention relates to compositions of Bis-(N,N disubstituted-aminophenyl)phenyl methane colorants that decolorize rapidly when applied to surfaces having a basic pH, such as concrete, but remain visually apparent on vegetation and soil. These compositions can also be used as pH indicators in their colorless form by developing color on acidic surfaces or in acidic materials, such as in/on paint, ice, concrete, and acid cured polymer systems. The compositions are also useful as spray pattern indicators for landscaping, construction, and golf course applications. The compositions also exhibit reduced toxicity when compared to other traditional colorants of similar shade used as spray pattern indicators.

### FIELD OF THE INVENTION

This invention relates to compositions of Bis-(N,N disubstituted-aminophenyl)phenyl methane colorants that decolorize rapidly when applied to surfaces having a basic pH, such as concrete, but remain visually apparent on vegetation and soil. These compositions can also be used as pH indicators in their colorless form by developing color on acidic surfaces or in acidic materials, such as in/on paint, ice, concrete, and acid cured polymer systems. The compositions are also useful as spray pattern indicators for landscaping, construction, and golf course applications. The compositions also exhibit reduced toxicity when compared to other traditional colorants of similar shade used as spray pattern indicators.

### BACKGROUND OF THE INVENTION

Spray pattern indicators provide a method for identifying a location to which a substance has been applied. Frequently, when a substance is applied to a location it may be difficult during the application process by virtue of, for instance, the nature of the substance and/or the size and configuration of the location to determine where the substance has been applied and where it has not been applied. For instance, where a substance such as a fertilizer, pesticide, etc., is being applied to a land area, e.g., farmland, golf course, rights-of-way, woodlands, etc., the appearance of the area which has been treated may not be sufficiently altered from the surrounding non-treated areas for the operator to avoid either overlapping which may be costly or even damaging to the location. Alternatively, certain areas may go untreated entirely so that the desired result of the application effort is not achieved in that area.

Solutions for the problem which have been suggested in the past have included the incorporation of certain non-fugitive, or permanent, dyes in the substance to be applied to identify the location to which the substance has been applied. In particular, it has been suggested, for instance, to incorporate certain non-fugitive dyes in pesticide compositions such as insecticides, fungicides, herbicides, etc. to identify the location to which the pesticide has been applied and to distinguish it in particular from those locations yet to be treated with the pesticide.

Unfortunately, there are numerous problems associated with such compositions containing permanent dyes. For instance, the dyes may be toxic or they may be incompatible with one or more ingredients of the composition. They may cause staining of workers' hands and clothing. Equipment may become stained. The permanent dyes which have been used typically show limited ability to biodegrade and, therefore, are subject to environmental objections such as, for instance, environmental objections associated with the contamination of streams and other bodies of water associated with “run-off” of aquatically toxic agricultural chemical water into those bodies of water. Further, and perhaps most importantly, it may be difficult to achieve a color that would be aesthetically acceptable from the viewpoint of the area being treated, for example, yellows and reds are virtually unobservable on green grass and unattractive on dormant grass.

A wide variety of fugitive, or non-permanent, colorants have been used to color code textiles during production and/or finishing operations to identify certain synthetic or natural fibers. Such fugitive colorants may be water fugitive, solvent fugitive or both water and solvent fugitive, although water fugitive colorants may be preferred according to the present invention, especially when the location may be an exterior location so that natural rainfall will wash away the initial coloration effect, thereby returning the location to its natural appearance. Colorants containing one or more polyethyleneoxy groups wherein the polyethyleneoxy group contains at least 2 repeating ethyleneoxy units in the molecule are generally considered water fugitive colorants; whereas colorants containing one or more propyleneoxy groups having similar repeating propyleneoxy units in the molecule are considered solvent fugitive.

Fugitive colorants which include polyethylene oxide colorants are described in U.S. Pat. No. 3,157,663 to Kuhn. Such colorants are a combination of a dyestuff radical and one or more polyethyleneoxy groups. Dyestuff radicals disclosed in the patent include nitroso, nitro, azo, diphenylmethane, triphenylmethane, xanthene, acridine, methine, thiazole, indamine, azine, oxazine, or anthraquinone radicals. Preferably, such radicals are attached to the polymeric constituents of the colorant compositions by an amino nitrogen.

Another type of fugitive colorant includes the alkaline-stable fugitive colorant of the triphenylmethane type as described in U.S. Pat. No. 3,927,044 to Foster et al. These colorants are alkaline stable due to the use of aromatic aldehydes containing an electron withdrawing substituent in the ortho position. They are considered alkaline stable if the colorant is capable of retaining its color in an alkaline solution, such as in a solution of sodium hydroxide at a pH of 11.

Yet another category of fugitive colorants are the ester capped alkyleneoxy fugitive colorants disclosed in U.S. Pat. No. 4,167,510 to Brendle. Such fugitive colorants comprise an organic dyestuff molecule having from 1 to 5 capped alkyleneoxy units wherein the total alkyleneoxy capped units in the molecule are from 2 to about 300. The alkylene moiety of the alkyleneoxy units contains from about 2 to 4 carbon atoms and the colorants of the invention can be made water and/or organic solvent soluble depending upon the particular capping moiety employed, the presence or absence of at least one ionic group and the total number of alkyleneoxy units present in the colorant molecule. The solubility, and thus the fugitivity of the colorants may be achieved irrespective of whether the relatively large dyestuff molecule is hydrophobic or hydrophilic.

Still another category of fugitive colorants includes those disclosed in U.S. Pat. No. 4,400,320 to Keller et al. These colorants are made from an aromatic compound containing between 6 and 20 carbon atoms and a linking moiety which are converted into a hydroxyalkylated compound. A further step includes the addition of a diazo compound to the hydroxyalkylated compound to form the final product. These colorants are red or purple in nature.

U.S. Pat. No. 5,520,943 to Brendle discusses the utilization of alkyleneoxy-substituted fugitive colorants for use as spray pattern indicators. These fugitive colorants are an improvement over permanent dyes in that they are washable from most surfaces including skin, textiles, and equipment. Additionally, these water soluble polyoxyalkylene substituted colorants are remarkably less toxic than dyes to aquatic organisms. However, on exposure to porous surfaces, such as concrete or brick, spray pattern indicators of this class can leave highly visible stains than may require days or weeks to fade. The presence of visual stains on these porous surfaces is unsightly and serves no purpose after the spray has been applied. Thus, there is a need for a polyoxy substituted colorant with reduced staining to skin, textiles, and equipment and reduced toxicity to aquatic organisms that does not leave visually apparent stains on porous surfaces, such as concrete or brick. Additionally, there is a need for colorless polyoxy substituted colorant precursors that generate color upon exposure to an acid surface or upon exposure to a medium that becomes acidic by generation of acid or removal of basic components.

It has been found that the present invention may overcome the visually apparent stains on porous surfaces, such as concrete or brick, associated with known processes involving, for instance, the use of substances containing staining permanent dyes or substances containing prior art fugitive colorants. Thus, it may now be possible to identify locations to which substances have been applied without causing visually apparent stains on porous surfaces, such as concrete or brick. It may further be possible to provide a process involving the use of a composition wherein the colorant component is (a) compatible with the components of the composition to be applied, (b) nontoxic, (c) biodegradable and, therefore, not harmful to the environment. In particular, it may be possible to provide a process employing marking colorants which are generally non-toxic to aquatic life especially fish when the colorant comes into contact with a given body of water as a result of “run-off” from the land areas in the watershed of that body of water.

In addition, it may be possible according to the process to achieve identification by coloration of locations to which a substance has been applied in such a way that the color can be provided in aesthetically pleasing shades of green and blue that look natural on green vegetation. This result may be especially desirable where the location to be treated is a turf grass such as that used in lawns, golf courses, athletic fields and the like that have concrete walkways or when spraying pesticides around the foundation of buildings. In addition, this pH sensitive color change can reveal coating inconsistencies where the coating and the substrate differ in pH, one being acidic and the other being neutral or basic and visa versa. This change of color can indicate neutral ice on top of basic concrete or basic paint on top of acid wall board. This effect is especially useful in clear coating visualization.

### BRIEF DESCRIPTION OF THE DRAWINGS

FIG. 1 is a line graph showing color change over time after application of various colorants to a concrete surface.

### DETAILED DESCRIPTION OF THE INVENTION

All U.S. and foreign patents and U.S. patent applications disclosed in this specification are hereby incorporated by reference in their entirety. For the purposes of this invention the term “fugitive” means a temporary colorant that can be removed easily by the processes of light exposure, exposure to rain or watering, or time.

This invention relates to a specific class of triphenylmethane (“TPM”) colorant compositions known as Bis-(N,N disubstituted-aminophenyl)phenyl methane colorants. TPM colorants consist of three aromatic rings linked by a central carbon atom. TPM colorants can be prepared by first condensing an aromatic aldehyde with two equivalents of an aromatic amine (which will hereafter be referred to as the “coupler” or “coupling component”) in the presence of an acid such as sulfuric acid, phosphoric acid, or muriatic acid. After condensation, the uncolored intermediate is oxidized using a variety of oxidizing agents (e.g. hydrogen peroxide, lead peroxide, chromium oxide, benzoylperoxide, benzoquione) to afford the TPM colorant. Variations in the substitution location and type on either the aldehyde or the coupler molecules can change the wavelength of light absorbed, thus providing a different color to the colorant species. The result of the substitution of groups in this manner is generally known to produce green and blue shades, but the fine shade variations produced are highly unpredictable. For example, the inclusion of oxygen-containing moieties tends to result in bluish shades, while exclusion of these moieties provides a fugitive colorant having a more green shade. Thus, a large amount of effort may be expended to find the right molecular combination to provide the most appropriate color shade for a given application.

The Bis-(N,N disubstituted-aminophenyl)phenyl methane colorants may be represented by the following general structure:

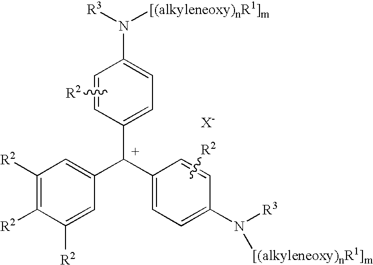

The Bis-(N,N disubstituted-aminophenyl)phenyl methane colorant compositions within the scope of the present invention contain phenyl rings that can be substituted with non-anionic charge containing groups including halides, alkyl groups, alkoxy groups, polyoxyalkylene groups, amido and sulphamino groups, hydrogen atoms, ester groups, and groups having methylene dioxy moieties linked to the 3 and 4 ring positions.

According to a preferred embodiment, the colorants of the present invention may be characterized by the following general formula (I):

3,4-diR²substituted phenyl-C⁺-{A-NB[(alkyleneoxy constituent)ₙR¹]ₘ}X⁻ (I)

wherein R² is a non-anionic charge containing group selected from halides, alkyl groups, alkoxy groups, polyoxyalkylene groups, amido and sulphamino groups, hydrogen atoms, ester groups, and groups having methylene dioxy moieties linked to the 3 and 4 ring positions; C is a carbon atom; A is a 1,4-linked R² substituted phenyl linking moiety; N is a nitrogen atom; B is selected from an (alkyleneoxy constituent)ₙR¹ group, a hydrogen atom, a C₁ to C₂₀ alkyl group, or an R² substituted aromatic group; the alkylene moiety of the alkyleneoxy constituent contains from about 2 to about 4 carbon atoms, a glycidol radical, or mixtures thereof; n is an integer of from about 2 to about 300; m is an integer of from about 1 to about 8; and R¹ is a member of the group consisting of sulfonates and sulfates of the substituent chains, hydrogen atoms, an alkyl substituted dicarboxylic acid or anhydride residue containing from 3 to about 22 carbon atoms, an alkyl radical containing up to about 20 carbon atoms, or a substituted radical of general formula (II):

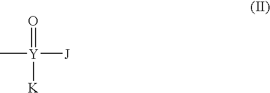

containing from 0 to about 20 carbon atoms, wherein Y is C, S, or P; O is an oxygen atom; J and K are independently selected from valence balancing electron pair, O⁻, OH, OM or OR³ wherein M is a cation moiety of an alkali metal, an alkaline earth metal, or ammonium and R³ is selected from an alkyl radical containing up to about 20 carbon atoms and an R² substituted aromatic radical; and wherein X⁻ is a counter ion selected from halides, sulfates, phosphates, carboxylates, carbonates, and counter ions covalently present in R¹.

It may be preferable that the R² group is selected from the group consisting of chloride, methoxy, polyoxyethylene, methyl, and methylene dioxy. It may also be preferable that R¹ and R² are dodecenyl succinate acid.

The term water soluble and/or fugitive as used herein is to be understood to mean that the colorant is substantially soluble in water and can be substantially removed by washing the location to which the substance containing the colorant has been applied with water. A typical example of washing operation would certainly include subjecting the location to rainwater, snow, etc. whereby the colorant may be removed. The term “water and organic solvent soluble and/or fugitive” is to be understood that the colorant is substantially soluble in water or an organic solvent and can be removed by washing the location with water or an organic solvent. Organic solvents which may be employed to treat the location are well-known in the art and include hydrocarbons, such as mineral oil, and organic solvents such as perchloroethylene, carbon tetrachloride, acetone, alcohol and the like.

The amount of alkyleneoxy-substituted fugitive colorant employed according to the process, that is the amount provided in association with the particular substance, will vary greatly depending upon the quantity of substance to be applied to a given area, the nature of the substance itself, the location to which the substance is to be applied and possibly other factors. In general the colorant will be employed in association with the substance in an amount sufficient to visually identify the area to which the substance has been applied. Of course the colorant may be employed in larger concentrations, for instance, in those applications where temporary coloration may be appropriate or desirable. In general upper levels of the concentration range for the colorant may be discouraged not by any performance disadvantages but rather by cost considerations. The actual amount of colorant employed may typically be at least about 0.2 ounce of colorant per 1000 square feet of location area to be applied. A preferred amount may be at least about 2.0 ounces.

According to the present invention a process is provided for temporarily identifying a location to which a substance has been applied, which comprises: (a) incorporating Bis-(N,N disubstituted-aminophenyl)phenyl methane colorant into a substance (e.g. a pesticide) prior to its application to a location (e.g. a porous substrate such as concrete) in an amount sufficient to identify the location to which the substance is to be applied, (b) thereafter applying the colorant-containing substance to the desired location, and (c) allowing the colorant to rapidly fade on the basic surface.

Locations to which a particular substance may be applied according to the present invention include virtually anything to which may be applied a given solid and/or liquid substance to typically achieve a desirable effect. According to a preferred embodiment of the invention, the location may be a given land area, e.g., agricultural land such as grazing land, crop land and the like; turf-covered land areas, e.g., lawns, golf courses, athletic fields, etc., and even other land areas, such as forests and highway and utility rights of way that have concrete areas that may be contacted with the colorant. Other locations which are contemplated by the invention include man-made surfaces, e.g., building surfaces such as foundations, concrete walkways, driveways and patios.

Substances which may be applied to a location according to the process of the present invention include any of a wide variety of liquid phase and/or solid phase substances that may achieve a desired effect when applied to the location. The amount of substance applied will be that amount that is conventionally applied to achieve a desired effect. In general the substance may be characterized in that the location to which the substance has been applied is not substantially changed in appearance immediately upon application of the substance to the location. Thus, it typically may be difficult, or at least inconvenient, for a person who is applying the substance by mechanical means or even by hand to determine where the substance has been applied to avoid overlapping or even indeed leaving certain areas entirely untreated. Overlapping may be costly depending upon the cost of the substance itself. Alternatively, if certain areas are left untreated by the substance the desired effect to be achieved by the substance may not come about. In addition, because of the presence of the color, it has been found that misapplication of the substance to areas which are not to be treated is more easily avoidable.

Substances which may be included within the scope of the present invention according to a preferred embodiment include, for instance, chemical compositions especially agricultural chemicals in solid and/or liquid phase, e.g., pesticide compositions especially fungicides, herbicides and insecticides; fertilizers (solid phase and/or liquid phase); and mixtures thereof. Additionally, the chemical compositions may be added to agricultural products used for hydroseeding which provides the aesthetically pleasing benefit of temporary green coloration on the ground where grass seed has been applied for some time until the grass begins to grow.

Examples of other types of chemicals not typically considered to be agricultural chemicals include sand or salt mixtures which may be applied to highways, sidewalks, etc. to alleviate hazardous conditions on snow and ice covered areas. Other uses include incorporation of the chemical composition into liquid or solid articles. For example, the composition may be incorporated into articles such as mosquito coils, which are popular repellents used in some countries heavily populated with mosquitoes. The coils are burned to release pesticide into the air which kills the mosquitoes. The chemical compositions are also ideal for use in wood products, such as, for example, fiberboard, as color indicators for various wood grades and densities.

It is also contemplated to be within the scope of this invention that a large variety of colors and shades may be obtained by blending the colorant composition of the present invention with one or more additional water soluble colorants. Blending of the colorants may be readily accomplished, for example, when combining colorants having substantially identical solubility characteristics. One exemplary class of colorants includes the Liquitint® colorants (available from Milliken Chemical of Spartanburg, S.C.). The Liquitint® colorants are generally water soluble, or dispersible, at room temperature and may be suitably blended with the colorant composition of the present invention to achieve improved colors and shades.

### EXAMPLES

The invention may be further understood by reference to the following examples which are not to be construed as limiting the scope of the present invention. For each of the colorants tested below, the absorbance was corrected to ensure equal color units per sample, i.e. equal color strength.

### A. Colorant Formulations and Preparation

### Colorant 1

105 grams of polyoxyethylene substituted aniline containing 10 mole equivalents of ethylene oxide (“EO”) was added to a reactor and charged with 9.5 grams benzaldhyde, 15 grams 32% hydrochloric acid, 2.2 grams urea, and 0.15 grams ammonium meta vanadate (“AMV”). The reactor was purged with nitrogen and then heated for 3 hours at 95° C. Oxidation was initiated by adding 41 grams 17.5% hydrogen peroxide (made by mixing 21 grams 35% hydrogen peroxide solution with 20 grams water) slowly at 95-100° C. The pH was adjusted to 4.0 by the addition of 25% sodium hydroxide. Water was removed by rotorary vacuum to give 118 grams of 95.94% solids dark bluish green liquid.

Several variations of this synthesis reaction are provided in Table 1 below and are intended to be exemplary teachings of the colorant compositions of the present invention. All process steps were the same as described above unless other wise noted in Table 1 below (i.e. reactor purge step, etc.). The absorbance (at 1 cm path length) of each sample was measured using a spectrophotometer; the results are provided as absorbtivity values where absorbtivity is a measure of absorbance per gram per liter (A/g/L).

TABLE 1
Synthesis of Bis-(N,N disubstituted-aminophenyl)phenyl methane
Colorant Compositions

| | | | | | | | | Solids |
|---|---|---|---|---|---|---|---|---|
| | | | | | | H₂O₂/ | Absorbtivity | (% wt/ |
| Sample | Aniline | Aldehyde | Acid | Urea | AMV | H₂O | (A/g/L) | wt) |
| Colorant 2 | 105 g | 12.58 g 4- | 15 g | 2.2 g | 0.15 g | 21 g/ | 31.46 | 95.94 |
| | 10EO | Chlorobenzaldehyde | Muriatic | | | 20 g | | |
| | | | Acid | | | | | |
| Colorant 3 | 105 g | 13.44 g Piperonal | 15 g | 2.2 g | 0.15 g | 21 g/ | 33.68 | 93.17 |
| | 10EO | (3,4-methylenedioxy | Muriatic | | | 20 g | | |
| | | benzaldehyde) | Acid | | | | | |
| Colorant 4 | 105 g | 10.76 g p-methyl | 15 g | 2.2 g | 0.15 g | 21 g/ | 33.28 | 95.27 |
| | 10EO | benzaldehyde | Muriatic | | | 20 g | | |
| | | | Acid | | | | | |
| Colorant 5 | 105 g | 38.69 g Vanillin 6EO | 15 g | 2.2 g | 0.15 g | 21 g/ | 22.21 | 95.02 |
| | 10EO | | Muriatic | | | 20 g | | |
| | | | Acid | | | | | |
| Colorant 6 | 105 g | 29.54 g p-hydroxy | 15 g | 2.2 g | 0.15 g | 21 g/ | 22.12 | 95.12 |
| | 10EO | 5EO | Muriatic | | | 20 g | | |
| | | | Acid | | | | | |
| Colorant 7 | 105 g | 9.5 g Benzaldehyde | 15 g | 2.2 g | 0.15 g | 21 g/ | 7.26 | 66.77 |
| | M-Tol | | Muriatic | | | 20 g | | |
| | 10EO | | Acid | | | | | |
| Colorant 8 | 61.67 g | 9.5 g Benzaldehyde | 15 g | 2.2 g | 0.15 g | 21 g/ | 21.46 | 57.92 |
| | 5EO | | Muriatic | | | 20 g | | |
| | | | Acid | | | | | |

### Colorant 9—Alkenylsuccinic ester acid

To a three neck round bottom flask, 200 grams of dry colorant derived by vacuum treatment of Colorant 8 was added followed by 266 grams dodecenylsuccinic anhydride (one equivalent anhydride per equivalent of hydroxyl end group). The solution was stirred and heated to 95° C. for 4 hours. The resulting dark green solution was washed with an equal volume of water two times to give 445 grams dark green dodecenyl succinic ester acid colorant of 95% solids, lambda max 630.5 and absorbtivity 33.46 A/g/L.

Color values for the Colorants described above are provided in Table 2 below. The color value was corrected for variability in water content by taking the absorbtivity value from Table 1 and dividing by the percent solids from Table 1 and multiplying this value by 100.

TABLE 2
Color Values for Synthesized
Bis-(N,N disubstituted-aminophenyl)phenyl methane
Colorant Compositions

| | | | Lambda | Color of |
|---|---|---|---|---|
| | | Color | Max Value | Sample |
| Sample | Chemical Name | Value | (Methanol) | Obtained |
| Colorant 1 | Phenyl-methyliumylidene-di(4,1- | 55.8 | 627 | Dark |
| | phenyleneamino)N,N-polyethyleneoxy(10), | | | Bluish |
| | Chloride | | | Green |
| Colorant 2 | 4-Chlorophenyl-methyliumylidene-di(4,1- | 32.79 | 633 | Green |
| | phenyleneamino)N,N-polyethyleneoxy(10), | | | |
| | Chloride | | | |
| Colorant 3 | 3,4-methylenedioxyphenyl-methyliumylidene- | 36.15 | 619 | Blue |
| | di(4,1-phenyleneamino)N,N- | | | |
| | polyethyleneoxy(10), Chloride | | | |
| Colorant 4 | 4-Methylphenyl-methyliumylidene-di(4,1- | 34.93 | 622 | Green |
| | phenyleneamino)N,N-polyethyleneoxy(10), | | | |
| | Chloride | | | |
| Colorant 5 | 3-Methoxy-4-Polyethyleneoxide(6)phenyl- | 23.37 | 615.5 | Blue |
| | methyliumylidene-di(4,1- | | | |
| | phenyleneamino)N,N-polyethyleneoxy(10), | | | |
| | Chloride | | | |
| Colorant 6 | 4-polyoxyethylene(5)phenyl- | 23.26 | 613.5 | Green |
| | methyliumylidene-di(4,1- | | | |
| | phenyleneamino)N,N-polyethyleneoxy(10), | | | |
| | Chloride | | | |
| Colorant 7 | Phenyl-methyliumylidene-di(4,1-(2- | 10.87 | 653.5 | Blue |
| | methyl)phenyleneamino)N,N- | | | |
| | polyethyleneoxy(10), Chloride | | | |
| Colorant 8 | Phenyl-methyliumylidene-di(4,1- | 37.05 | 628 | Bluish |
| | phenyleneamino)N,N-polyethyleneoxy(5), | | | Green |
| | Chloride | | | |
| Colorant 9 | Phenyl-methyliumylidene-di(4,1- | 35.22 | 630.5 | Dark |
| | phenyleneamino)N,N-polyethyleneoxy(5)- | | | Green |
| | tetrakisDodecenylsuccinate acid, inner salt | | | |

### B. Comparative Example Description

Several commercially available colorant compositions were also purchased for evaluation. These colorants are notated as Comparative Examples 1-4 below.
COMPARATIVE COLORANT 1—“Blazon® Blue Spray Pattern Indicator”, an ortho sulfonate phenyl TPM colorant synthesized from poly(ethyleneoxy) substituted aniline as described in U.S. Pat. No. 3,927,044 to Foster et al., available from Milliken & Company of Spartanburg, S.C.
COMPARATIVE COLORANT 2—“Hi Vis Bullseye® Spray Pattern Indicator”, a non-staining violet liquid colorant designed to be used with pesticide, fertilizer and/or plant growth regulator tank mixes; contains a triphenylmethane colorant synthesized from poly(ethyleneoxy) substituted aniline and a disubstituted aminobenzaldehyde; available from Milliken & Company of Spartanburg, S.C.
COMPARATIVE COLORANT 3—“Acid Blue 9”, an FDA certified food dye having Color Index No. 42090 and having the chemical formula C₃₇H₃₄N₂O₉S₃2Na; a phenyl substituted TPM derived from o-formylbenzenesulfonate.
COMPARATIVE COLORANT 4—“Basic Green 4”, also known as malachite green; a N,N-dimethyl aminophenylphenyl methane dye having Color Index No. 42000 and having the chemical formula C₂₃H₂₅N₂Cl; a basic, non-fugitive (i.e. staining) dye often used for dyeing textiles.

### C. Test Methods and Evaluation

Test 1: DE CMC Color Evaluation
Test 2: Lightfastness Test
Test 3: Test for pH Effect on De-Coloration
Test 4: Skin Staining Test
Test 5: Cellulose Powder Staining Test

### TEST 1: DE CMC Color Evaluation

In order to determine the de-coloration rate over time of the colorant compositions of the present invention, the colorants were subjected to color evaluation testing. A measurement of the relative color of the substrates can be determined by DE CMC (i.e. delta E CMC) values. DE CMC values provide a measure of the overall color difference for all uniform color spaces, where DE CMC values represent the magnitude of difference between a color and a reference (in this case, a pure white standard). The higher the DE CMC value, the more pronounced the difference in color. Said another way, smaller DE CMC values represent colors that are closer to white. A spectrophotometer is used to calculate DE CMC values based on wavelength and reflectance data for each sample.

Sample Preparation and Test Method:

### A. Sample Preparation

Each sample was prepared by adding 0.5 parts of each colorant to 64 parts liquid weed killer (Round-Up® Ready to Use Plus) at room temperature. The absorbance of each colorant was corrected to ensure equal color units per sample. The samples were inverted to mix. The following samples were thus created and tested:
Inventive Example 1: Colorant 1 in weed killer
Comparative Example 1: Comparative Colorant 1 in weed killer
Comparative Example 2: Comparative Colorant 2 in weed killer
Comparative Example 3: Comparative Colorant 3 in weed killer

### B. Test Method

Each sample was sprayed on a cement surface and monitored to determine color deterioration by delta E (“ΔE” or “DE”). Pump sprayers were obtained from Round-Up® bottles for the application of solution on the cement surface. Using these sprayers attached to the colored Round-Up® solutions, two strokes were sprayed in a sweeping motion onto the cement surface.

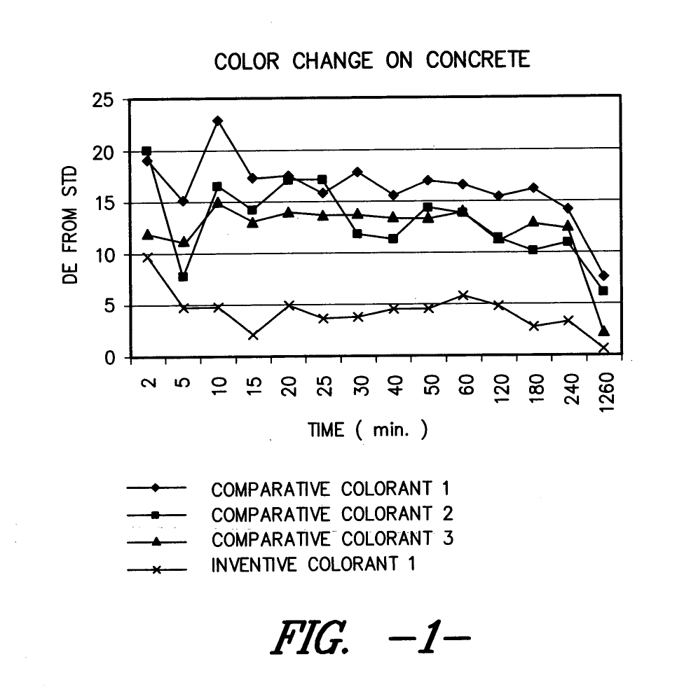

A portable X-Rite 938 Spectrodensitometer was used to take L,a,b color measurements and calculate DE CMC values at a range of time intervals from the initial application up to 21 hours. Inventive Example 1 was compared to Comparative Examples 1-3. The test results are shown in Table 3A and 3B and FIG. 1. The “Uncolored Standard” is the DE CMC values for the cement surface with no colorant applied. Thus, it represents the background color or the control in this test.

Table 3B provides the same data from Table 3A, except that the values for the Uncolored Standard have been subtracted from the values provided in Table 3A. “N/a” indicates that there was no data for that time period.

TABLE 3A
Comparison of Magnitude of Color Difference
as Determined by DE CMC Values

| | | Com- | Com- | | |
|---|---|---|---|---|---|
| | | parative | parative | Comparative | Inventive |
| | | Example 1 | Example 2 | Example 3 | Example 1 |
| TIME | Uncolored | DE CMC | DE CMC | DE CMC | DE CMC |
| (Minutes) | Standard | Value | Value | Value | Value |
| 2 | 16.5 | 35.6 | 36.41 | 28.31 | 26.17 |
| 5 | 17.35 | 32.5 | 24.99 | 28.65 | 22.11 |
| 10 | 4.27 | 27.09 | 20.67 | 19.17 | 9.13 |
| 15 | 3.35 | 20.71 | 17.45 | 16.41 | 5.55 |
| 20 | 2.05 | 19.49 | 19.13 | 15.97 | 6.93 |
| 25 | 2.61 | 18.41 | 19.65 | 16.29 | 6.25 |
| 30 | 2.39 | 20.23 | 14.13 | 16.21 | 6.19 |
| 40 | 2.01 | 17.51 | 13.21 | 15.39 | 6.55 |
| 50 | 2.75 | 19.73 | 17.03 | 16.15 | 7.32 |
| 60 | 1.87 | 18.41 | 15.63 | 15.89 | 7.67 |
| 120 | 3.49 | 18.87 | 14.81 | 14.69 | 8.25 |
| 180 | 2.11 | 18.25 | 12.21 | 15.03 | 4.91 |
| 240 | 1.91 | 16.03 | 12.75 | 14.35 | 5.23 |
| 1260 | 4.81 | 12.39 | 10.87 | 7.11 | 5.43 |

TABLE 3B
Comparison of Magnitude of Color Difference
as Determined by DE CMC Values With Background Values Removed

| | Comparative | Comparative | Comparative | Inventive |
|---|---|---|---|---|
| | Example 1 | Example 2 | Example 3 | Example 1 |
| TIME | DE CMC | DE CMC | DE CMC | DE CMC |
| (Minutes) | Value | Value | Value | Value |
| 2 | 19.1 | 19.91 | 11.81 | 9.67 |
| 5 | 15 | 7.49 | 11.15 | 4.61 |
| 10 | 22.82 | 16.4 | 14.9 | 4.86 |
| 15 | 17.36 | 14.1 | 13.06 | 2.2 |
| 20 | 17.44 | 17.08 | 13.92 | 4.88 |
| 25 | 15.8 | 17.04 | 13.68 | 3.64 |
| 30 | 17.84 | 11.74 | 13.82 | 3.8 |
| 40 | 15.5 | 11.2 | 13.38 | 4.54 |
| 50 | 16.98 | 14.28 | 13.4 | 4.57 |
| 60 | 16.54 | 13.76 | 14.02 | 5.8 |
| 120 | 15.38 | 11.32 | 11.2 | 4.76 |
| 180 | 16.14 | 10.1 | 12.92 | 2.8 |
| 240 | 14.12 | 10.84 | 12.44 | 3.32 |
| 1260 | 7.58 | 6.06 | 2.3 | 0.62 |

The results shown in Table 3B and FIG. 1 indicate that Inventive Example 1 more quickly approached loss of color (i.e. a DE CMC value of zero) than did the Comparative Examples.

### TEST 2: Lightfastness Test

In order to determine the effect of ultraviolet light on the de-coloration rate over time of the colorant compositions of the present invention, the colorants were subjected to a lightfastness test. The test was performed according to AATCC Test Method 16-1993 Option E. Comparative Colorants 1-4 were also tested.

The results are provided in Tables 4A and 4B. Comparative Colorant is notated as “Comp. Colorant.”

Table 4A shows the measurements taken by spectrophotometry. The results are provided as DE CMC values and represent the color difference from the standard or control, which was white paper. Table 4B shows the percent fade calculated by taking the initial DE CMC value (at time zero) from Table 4A and subtracting the DE CMC value at each of the time intervals from Table 4A. This value for each of the time intervals is then divided by the initial DE CMC value and multiplied by 100. The final value represents the percent color fade exhibited by the sample.

TABLE 4A
Effect of Ultraviolet Light on Colorants as Determined by DE CMC Values

| Exposure Time | | | | | Comp. | Comp. | Comp. | Comp. |
|---|---|---|---|---|---|---|---|---|
| (hours) | Colorant 3 | Colorant 4 | Colorant 5 | Colorant 8 | Colorant 1 | Colorant 2 | Colorant 3 | Colorant 4 |
| 0 | 75.15 | 50.95 | 38.47 | 51.09 | 55.09 | 57.59 | 58.19 | 44.71 |
| (initial) | | | | | | | | |
| 2 | 36.95 | 41.31 | 32.17 | 39.83 | 49.89 | 54.61 | 52.89 | 14.37 |
| 5 | 34.25 | 38.99 | 31.67 | 38.01 | 45.27 | 56.93 | 49.83 | 13.05 |
| 10 | 33.39 | 37.19 | 29.83 | 12.29 | 42.27 | 34.93 | 46.51 | 9.65 |
| 20 | 29.45 | 30.97 | 7.15 | 8.95 | 34.19 | 27.27 | 40.21 | 5.67 |

TABLE 4B
Effect of Ultraviolet Light on Colorants - Percent Color Fade Values

| Exposure Time | | | | | Comp. | Comp. | Comp. | Comp. |
|---|---|---|---|---|---|---|---|---|
| (hours) | Colorant 3 | Colorant 4 | Colorant 5 | Colorant 8 | Colorant 1 | Colorant 2 | Colorant 3 | Colorant 4 |
| 0 | 0 | 0 | 0 | 0 | 0 | 0 | 0 | 0 |
| 2 | 50.83 | 18.92 | 16.38 | 22.04 | 9.44 | 5.18 | 9.11 | 67.86 |
| 5 | 54.42 | 23.47 | 17.68 | 25.60 | 17.83 | 1.15 | 14.37 | 70.81 |
| 10 | 55.57 | 27.01 | 22.46 | 75.94 | 23.27 | 39.35 | 20.07 | 78.42 |
| 20 | 60.81 | 39.22 | 81.42 | 82.48 | 37.94 | 52.65 | 30.90 | 87.32 |

### TEST 3: Test for pH Effect on De-Coloration

In order to show the effect of pH on de-coloration, or color loss, of the colorant compositions of the present invention, the colorants were subjected to the following test procedures.

### A. Sample Preparation and Test Method—Absorbance Values

Several 1% solutions of colorant were prepared and added to standard aqueous pH buffer solutions available from VWR and Aldrich to give absorbance of less than one. The colorant solution was tested for absorbance using a DU 7 Spectrophotometer.

The test results are provided in Table 5. In general, the pKa value represents the pH value when approximately half of the colorant is converted to its colorless form. “N/a” indicates that there was no data for that pH value. “Comparative” is indicated as “Comp.”

TABLE 5
Effect of pH Change on Absorbance

| pH | | | | | Comp. | Comp. | Comp. | Comp. |
|---|---|---|---|---|---|---|---|---|
| Value | Colorant 1 | Colorant 3 | Colorant 4 | Colorant 5 | Colorant 1 | Colorant 2 | Colorant 3 | Colorant 4 |
| 2 | 0.95 | 0.9589 | 0.8881 | 0.8266 | 0.8345 | 0.7128 | 0.764 | 0.319 |
| 4 | 0.95 | 0.955 | 0.8878 | 0.8269 | 0.8425 | 0.7545 | 0.7699 | 0.3843 |
| 6 | 0.82 | 0.9609 | 0.8304 | 0.7735 | 0.8237 | 0.7245 | 0.7253 | 0.4546 |
| 7 | 0.35 | 0.7309 | 0.569 | 0.5674 | n/a | n/a | n/a | 0.2678 |
| 8 | 0.05 | 0.2375 | 0.154 | 0.1928 | 0.8064 | 0.7351 | 0.7829 | 0.0538 |
| 9 | 0.005 | 0.0424 | 0.0244 | 0.0527 | n/a | n/a | n/a | 0.0361 |
| 10 | 0.005 | 0.0199 | 0.0079 | 0.0312 | 0.7923 | 0.7472 | 0.804 | 0.0111 |
| pKa | 6.6 | 7.5 | 7.3 | 7.3 | >10 | >10 | >10 | 7 |
| Value | | | | | | | | |
| Lambda | 618 | 619 | 622 | 615 | 629 | 592.5 | 629 | 617 |
| Max | | | | | | | | |
| Value | | | | | | | | |
| (Water) | | | | | | | | |

The results shown in Table 5 indicate that as the inventive Colorants were exposed to more basic conditions (i.e. higher pH values), the Colorants became nearly colorless. In contrast, the Comparative Colorants were also tested by the same method and showed no color loss as the pH value changed, except for Comparative Example 4, i.e. Basic Green 4. Comparative Example 4 is a Bis-(N,Ndimethylaminophenyl)phenyl TPM colorant, a non-fugitive dye.

Additionally, the test results show that the pKa value of the inventive Colorants is between about 6.6 and 7.5. Thus, the inventive, fugitive colorants have an approximate pKa value of between about 6 and about 8.

### B. Sample Preparation and Test Method—Latex Paint Example

A paint sample was prepared by adding 1 part of Colorant 1 to 99 parts of white latex paint (Severe Weather® paint by Valspar). The sample was stirred to mix. With continuous stirring, ammonium hydroxide was added to the paint sample until it turned white.

A paint brush was used to apply a thin layer of the white modified paint sample to the surface of an untreated piece of Southern Yellow Pine wood. The color change was monitored as the paint dried on the surface. Initially, the paint was white, but within minutes, as the alkaline surface became more acidic in nature, the green color from Colorant 1 returned as the paint dried.

### C. Sample Preparation and Test Method—Ice Example

A solution of Colorant 1 was made by adding 1 part of Colorant 1 to 99 parts of water at room temperature. The solution was shaken to mix and put into a spray bottle for application. A concrete brick was covered with dry ice and allowed to cool. Room temperature water was sprayed on half the brick in order to create a layer of ice. Water was then repeatedly sprayed on the ice layer essentially building up the ice to create a slope on one end of the brick. The brick was again covered with dry ice to ensure that the brick was frozen throughout.

After being removed from the dry ice, the brick was sprayed evenly with the solution that contained 1 percent Colorant 1. The cold brick was allowed to thaw at room temperature and the presence of color was monitored on the surface over the next 2 hours. The color from the Colorant 1 solution remained on the surface of the ice and disappeared from the concrete surface.

### Test 4: Skin Staining Test

In order to illustrate the staining/non-staining properties of some of these colorants and dyes described herein, a simple test was performed on human skin.

Comparative Colorant 4 (Basic Green 4), Inventive Colorant 1 (10 ethylene oxide monomers on each of two Aniline components), and Inventive Colorant 8 (5 ethylene oxide monomers on each of two Aniline components) were each separately dissolved in water to give an equal absorbtivity of 22 A/g/L. A cotton swab was saturated with a selected colored solution and applied to the palm of a volunteer in a stripe approximately ¼″ by 2″. For each color, this application was repeated two times directly over the first application stripe. The hand was then allowed to dry for 15 minutes and was then washed with liquid Ivory® soap and warm water.

The color depth of each stripe on the skin was visually evaluated before and after washing. The following visual rating scale was used to evaluate the staining/non-staining attribute of each color: 1=no change from pre-washed color, 2=slight removal of color, 3=removal of about 50% of color, 4=less than 25% color remaining, 5=slight to no stain remaining. Test results are provided in Table 6.

TABLE 6
Results of Skin Staining Test

| | Rating Scale |
|---|---|
| Sample | Value |
| Colorant 1 | 5 |
| (20 EO) | |
| Colorant 8 | 5 |
| (10 EO) | |
| Comparative | 3 |
| Colorant 4 | |

The results in Table 6 illustrate the non-staining, fugitive property of the colorants of the present invention. Furthermore, the staining, non-fugitive property of Comparative Colorant 4 (Basic Green 4) is also demonstrated. Thus, it is clear that the colorants of the present invention are substantially less staining to skin than the N,N Dimethylamino phenyl TPM version derived from the same Benzaldehyde (i.e. Basic Green 4).

### Test 5: Cellulose Powder Staining Test

Two of the inventive Colorants were evaluated for staining on cellulose powder according to the procedure described below.

Colorant 8 was diluted in water to an absorbtivity value of 33.5 A/g/L; then 0.73 parts was dissolved in 5 parts of methanol to give a stock solution A. One part of this solution was mixed with 10 parts of cellulose microcrystalline powder available from Aldrich. The colored powder was dried in a beaker in a 100° C. oven over 20 minutes with occasional stirring. The dry powder was cooled and stirred with 50 parts deionized water for one minute. The slurry was filtered and the filtrate was collected. The percent retention of color on the cellulose powder was calculated from the ratio of the absorbance of one part stock solution A diluted with 50 parts water and the absorbance of the filtrate solution.

The same procedure was used with a stock solution of 0.73 parts dodecenyl succinic ester acid colorant, Inventive Colorant 9, dissolved in 5 ml of methanol. The percent retention of color was determined in the same manner.

TABLE 7
Cellulose Powder Staining Test

| | Percent Color |
|---|---|
| Sample | Retention |
| Cellulose Powder Colored with | 56 |
| Colorant 8 | |
| Cellulose Powder Colored with | 92.6 |
| Colorant 9 | |

The results in Table 7 indicate that substitution of the inventive Colorants with alkyl groups substantially increases the retention of color on cellulosic powder. This modification provides colorants that exhibit greater resistance to rain and also exhibit reduction in colored run-off to water bodies, such as streams and lakes.

Thus, the above description and examples show that Bis-(N,N disubstituted-aminophenyl)phenyl methane colorants are useful as indicators having the desired characteristics of temporary surface coloration, low toxicity, and ease of application. As has been described herein, the colorant composition possesses a significant advantage over current products, in that it exhibits good color upon initial application to a surface, yet quickly de-colorizes after exposure to ultraviolet light and/or after exposure to a high pH environment and is less toxic than some current commercial products.

These and other modifications and variations to the present invention may be practiced by those of ordinary skill in the art, without departing from the spirit and scope of the present invention. Furthermore, those of ordinary skill in the art will appreciate that the foregoing description is by way of example only, and is not intended to limit the scope of the invention described in the appended claims.

We claim:

1. A pH sensitive, fugitive colorant composition characterized in that the colorant composition exhibits color in an acidic or neutral pH range and exhibits loss of color in an alkaline pH range, wherein the colorant is represented by general formula (I):
3,4-diR²substituted phenyl-C⁺-{A-NB[(alkyleneoxy constituent)ₙR¹]ₘ}X⁻ (I)
wherein R² is a non-anionic charge containing group selected from halides, alkyl groups, alkoxy groups, polyoxyalkylene groups, amido and sulphamino groups, hydrogen atoms, ester groups, and groups having methylene dioxy moieties linked to the 3 and 4 ring positions; C is a carbon atom; A is a 1,4-linked R² substituted phenyl linking moiety; N is a nitrogen atom; B is selected from an (alkyleneoxy constituent)ₙR¹ group, a hydrogen atom, a C₁ to C₂₀ alkyl group, or an R² substituted aromatic group;
the alkylene moiety of the alkyleneoxy constituent contains from about 2 to about 4 carbon atoms, a glycidol radical, or mixtures thereof; n is an integer of from about 2 to about 300; m is an integer of from about 1 to about 8; and R¹ is a member of the group consisting of sulfonates and sulfates of the substituent chains, hydrogen atoms, an alkyl substituted dicarboxylic acid or anhydride residue containing from 3 to about 22 carbon atoms, a radical containing up to about 20 carbon atoms, or alkyl substituted radical of general formula (II): 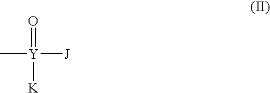
containing from 0 to about 20 carbon atoms, wherein Y is C, S, or P; O is an oxygen atom; J and K are independently selected from valence balancing electron pair, O⁻, OH, OM or OR³ wherein M is a cation moiety of an alkali metal, an alkaline earth metal, or ammonium and R³ is selected from an alkyl radical containing up to about 20 carbon atoms and an R² substituted aromatic radical; and wherein X⁻ is a counter ion selected from halides, sulfates, phosphates, carboxylates, carbonates, and counter ions covalently present in R¹.

2. The colorant composition of claim 1, wherein the colorant has the following general structure:

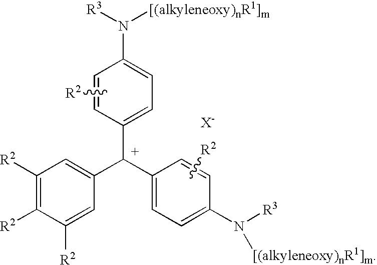

3. The colorant composition of claim 1, wherein the colorant is water soluble.

4. The colorant composition of claim 3, wherein the alkyleneoxy constituent is selected from the group consisting of ethyleneoxy and propyleneoxy units.

5. The colorant composition of claim 4, wherein the colorant comprises a polyethyleneoxy group having at least two repeating ethyleneoxy units.

6. The colorant composition of claim 1, wherein R² is selected from the group consisting of chloride, methoxy, polyoxyethylene, methyl, and methylene dioxy.

7. The colorant composition of claim 1, wherein R¹ and R² are dodecenyl succinate acid.

8. The colorant composition of claim 1, wherein the colorant a liquid.

9. The colorant composition of claim 8, wherein the liquid material is paint.

10. The colorant composition of claim 1, wherein the colorant is combined with at least one agricultural chemical to form an agricultural chemical product, wherein the agricultural chemical is selected from the group consisting of insecticides, fungicides, herbicides, fertilizers and mixtures thereof.

11. The colorant composition of claim 10, wherein the agricultural chemical product is applied to agricultural land, turf-covered land, and forest areas in an amount sufficient to identify an area to which the agricultural chemical product has been applied.

12. The colorant composition of claim 1, wherein the colorant is blended with at least one additional colorant.

13. The colorant composition of claim 1, wherein the colorant exhibits a percent color fade of:
(a) between about 15% and about 60% after 2 hours of exposure to ultraviolet light, and
(b) between about 40% and about 85% after 20 hours of exposure to ultraviolet light,
wherein the percent color fade is calculated from measurements taken according to AATCC Test Method 16-1993 Option E.

14. The colorant composition of claim 1, wherein the colorant exhibits absorbance values of:
(a) at least about 0.8 at a pH value of 2, and
(b) less than about 0.1 at a pH value of 10,
wherein absorbance is determined by spectrophotometry measurements.

15. The colorant composition of claim 14, wherein the colorant exhibits a pKa value of between about 6 and about 8.

16. The colorant composition of claim 1, wherein the colorant is incorporated into a solid material.

17. The colorant composition of claim 16, wherein the solid material is a mosquito coil.

18. The colorant composition of claim 16, wherein the solid material is a wood product.

19. The colorant composition of claim 18, wherein the wood product is fiberboard.

20. A process for identifying the location to which a chemical composition has been applied comprising incorporating an alkyleneoxy substituted fugitive colorant into the composition, prior to its application to the location, in an amount sufficient to identify the location, wherein the colorant exhibits color in an acidic or neutral pH range and exhibits loss of color in an alkaline pH range, and wherein the colorant is represented by general formula (I):
3,4-diR²substituted phenyl-C⁺-{A-NB[(alkyleneoxy constituent)ₙR¹]ₘ}X⁻ (I)
wherein R² is a non-anionic charge containing group selected from halides, alkyl groups, alkoxy groups, polyoxyalkylene groups, amido and sulphamino groups, hydrogen atoms, ester groups, and groups having methylene dioxy moieties linked to the 3 and 4 ring positions; C is a carbon atom; A is a 1,4-linked R² substituted phenyl linking moiety; N is a nitrogen atom; B is selected from an (alkyleneoxy constituent)ₙR¹ group, a hydrogen atom, a C₁ to C₂₀ alkyl group, or an R² substituted aromatic group; the alkylene moiety of the alkyleneoxy constituent contains from about 2 to about 4 carbon atoms, a glycidol radical, or mixtures thereof; n is an integer of from about 2 to about 300; m is an integer of from about 1 to about 8; and R¹ is a member of the group consisting of sulfonates and sulfates of the substituent chains, hydrogen atoms, an alkyl substituted dicarboxylic acid or anhydride residue containing from 3 to about 22 carbon atoms, an alkyl radical containing up to about 20 carbon atoms, or a substituted radical of general formula (II): 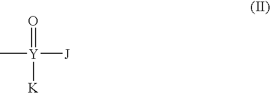
containing from 0 to about 20 carbon atoms, wherein Y is C, S, or P; O is an oxygen atom; J and K are independently selected from valence balancing electron pair, O⁻, OH, OM or OR³ wherein M is a cation moiety of an alkali metal, an alkaline earth metal, or ammonium and R³ is selected from an alkyl radical containing up to about 20 carbon atoms and an R² substituted aromatic radical; and wherein X⁻ is a counter ion selected from halides, sulfates, phosphates, carboxylates, carbonates, and counter ions covalently present in R¹.

21. The process of claim 20, wherein the alkyleneoxy substituted fugitive colorant has the following general structure:

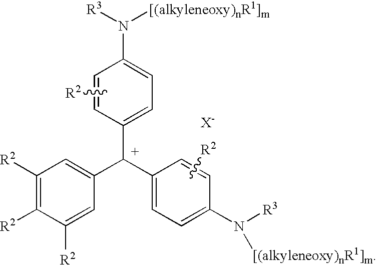

22. The process of claim 20, wherein the alkyleneoxy substituted fugitive colorant is water soluble.

23. The process of claim 22, wherein the alkyleneoxy constituent is selected from the group consisting of ethyleneoxy and propyleneoxy units.

24. The process of claim 23, wherein the alkyleneoxy substituted fugitive colorant comprises a polyethyleneoxy group having at least two repeating ethyleneoxy units.

25. The process of claim 20, wherein R² is selected from the group consisting of chloride, methoxy, polyoxyethylene, methyl, and methylene dioxy.

26. The process of claim 20, wherein R¹ and R² are dodecenyl succinate acid.

27. The process of claim 20, wherein the composition is a liquid.

28. The process of claim 27, wherein the liquid is paint.

29. The process of claim 20, wherein the composition further includes at least one agricultural chemical selected from the group consisting of insecticides, fungicides, herbicides, fertilizers, and mixtures thereof.

30. The process of claim 20, wherein the location is selected from the group consisting of agricultural land, turf-covered land and forests.

31. A process for altering the color of an article comprising:
(a) incorporating a colorant composition into the article, wherein the colorant composition is represented by general formula (I):
3,4-diR²substituted phenyl-C⁺-{A-NB[(alkyleneoxy constituent)ₙR¹]ₘ}X⁻ (I)
wherein R² is a non-anionic charge containing group selected from halides, alkyl groups, alkoxy groups, polyoxyalkylene groups, amido and sulphamino groups, hydrogen atoms, ester groups, and groups having methylene dioxy moieties linked to the 3 and 4 ring positions; C is a carbon atom; A is a 1,4-linked R² substituted phenyl linking moiety; N is a nitrogen atom; B is selected from an (alkyleneoxy constituent)ₙR¹ group, a hydrogen atom, a C₁ to C₂₀ alkyl group, or an R² substituted aromatic group; the alkylene moiety of the alkyleneoxy constituent contains from about 2 to about 4 carbon atoms, a glycidol radical, or mixtures thereof; n is an integer of from about 2 to about 300; m is an integer of from about 1 to about 8; and R¹ is a member of the group consisting of sulfonates and sulfates of the substituent chains, hydrogen atoms, an alkyl substituted dicarboxylic acid or anhydride residue containing from 3 to about 22 carbon atoms, an alkyl radical containing up to about 20 carbon atoms, or a substituted radical of general formula (II): 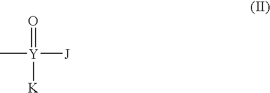
containing from 0 to about 20 carbon atoms, wherein Y is C, S, or P; O is an oxygen atom; J and K are independently selected from valence balancing electron pair, O⁻, OH, OM or OR³ wherein M is a cation moiety of an alkali metal, an alkaline earth metal, or ammonium and R³ is selected from an alkyl radical containing up to about 20 carbon atoms and an R² substituted aromatic radical; and wherein X⁻ is a counter ion selected from halides, sulfates, phosphates, carboxylates, carbonates, and counter ions covalently present in R¹;
(b) adjusting the pH of the colorant-containing article such that in an acidic or neutral pH range the colorant-containing article exhibits color and in an alkaline pH range the colorant-containing article exhibits loss of color.

32. The process of claim 31, wherein the colorant composition has the following general structure:

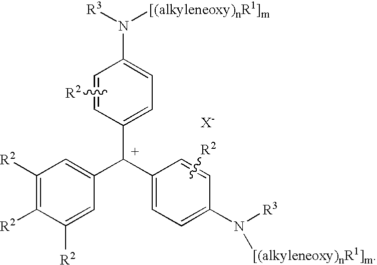

33. The process of claim 31, wherein the colorant-containing article is a liquid.

34. The process of claim 33, wherein the liquid is paint.

35. The process of claim 31, wherein the colorant-containing article is a solid material.

36. The process of claim 35, wherein the solid material is a mosquito coil.

37. The process of claim 35, wherein the solid material is a wood product.

38. The process of claim 37, wherein the wood product is fiberboard.
